(19) United States
(12) Patent Application Publication
(10) Pub. No.: US 2018/0199879 A1
(43) Pub. Date: Jul. 19, 2018

(54) System and method to control temperature

(71) Applicant: Peter Kanistros, Mars, PA (US)

(72) Inventor: Peter Kanistros, Mars, PA (US)

(21) Appl. No.: 15/872,476

(22) Filed: Jan. 16, 2018

Related U.S. Application Data
(60) Provisional application No. 62/447,379, filed on Jan. 17, 2017.

(51) Int. Cl. A61B 5/00; A61N 5/06; A61F 7/10

(52) U.S. Cl. CPC A61B 5/448; A61F 2007/0004; A61F 7/10; A61N 5/0617

(57) ABSTRACT

Disclosed is a device and method of controlling blood flow to a selected area. Also disclosed is a system and method to regulate a temperature at a selected location. The system and method may be applied to a subject for a selected period of time.

### CROSS-REFERENCE TO RELATED APPLICATIONS

This application claims the benefit of U.S. Provisional Application No. 62/447,379, filed on Jan. 17, 2017. The entire disclosure of the above application is incorporated herein by reference.

### FIELD

The subject disclosure relates to hair loss prevention therapy, and particularly to a method and system of thermal regulation.

### BACKGROUND

This section provides background information related to the present disclosure which is not necessarily prior art.

In various procedures, such as treating selected diseases in a living subject, side effects may occur. Various individuals may desire to reduce certain side effects. In some individuals administering certain chemotherapy drugs as a treatment for cancers may cause hair growth to be limited or stopped. In some instances, certain devices, such as those disclosed in U.S. Pat. No. 9,101,463 and U.S. Pat. No. 9,421,125, may be placed on a patient's head to assist in regulating a temperature of a portion of the patient's head.

### SUMMARY

This section provides a general summary of the disclosure, and is not a comprehensive disclosure of its full scope or all of its features.

Disclosed is a system that is configured to cool and/or maintain a selected temperature of at least a surface of a scalp and/or to a selected depth of dermis.

In addition the system and method includes limiting blood flow to selected regions of the head, including the scalp, at least by restricting and reducing blood flow through selected arteries into the head. For example, restriction or pressure application members may engage or be applied to arteries at or near a patient's head. Pressure application members may be placed at substantially specific locations, adjusted to be at specific locations, or positioned at known and selected placements around a circumference of a patient's head. In various embodiments, the pressure application members may be positioned to apply pressure at or near both occipital arteries and temporal arteries on the patient's head to mechanically restrict or apply pressure to the arteries to restrict flow of blood into the head.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic illustration of a regulating device, according to various embodiments;

FIG. 2 is a schematic illustration of the device of FIG. 1 on a subject;

FIG. 3A is a side view of a subject wearing a regulating device, according to various embodiments;

FIG. 3B is an anterior view of a subject wearing the device of FIG. 3A;

FIG. 3C is a posterior view of a subject wearing the device of FIG. 3A;

FIG. 4 is an exploded environmental view of the device of FIG. 3A;

FIG. 5A is an exploded view of the device of FIG. 3A;

FIG. 5B is a perspective assembled view of the device of FIG. 3A;

FIG. 6 is a detailed view of a medium transfer tubing;

FIG. 7A is a detailed perspective view of a compression device of FIG. 3A;

FIG. 8 is a detailed view of a bumper member assembly, according to various embodiments;

FIG. 9A is a detailed view of the compression device and bumper member assembly;

FIG. 9B is a detailed view of a bumper of the compression device and bumper member assembly;

FIG. 10 is a schematic view of a regulating device and pump assembly; and

FIG. 11 is a cross-sectional view of a schematic of a regulating device, according to various embodiments.

Corresponding reference numerals indicate corresponding parts throughout the several views of the drawings.

### DETAILED DESCRIPTION

Example embodiments will now be described more fully with reference to the accompanying drawings.

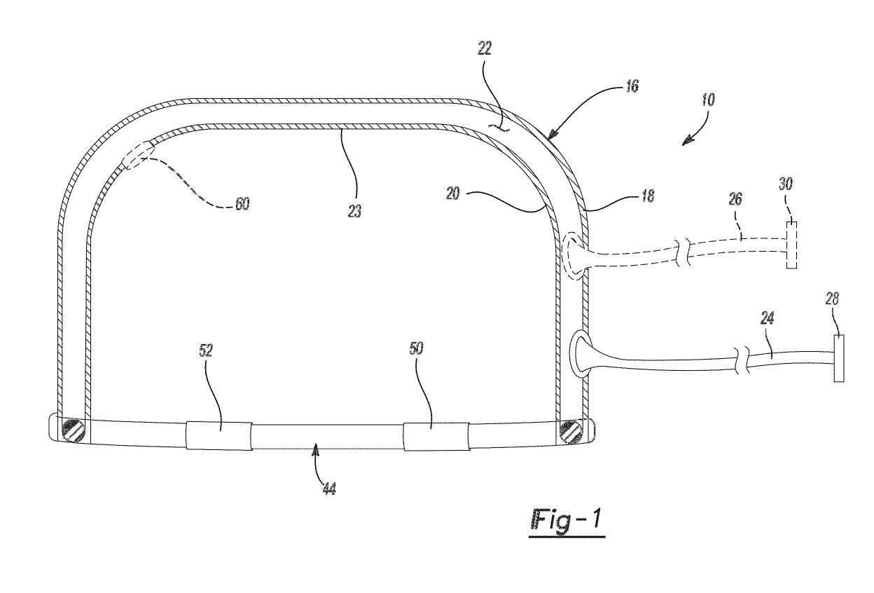

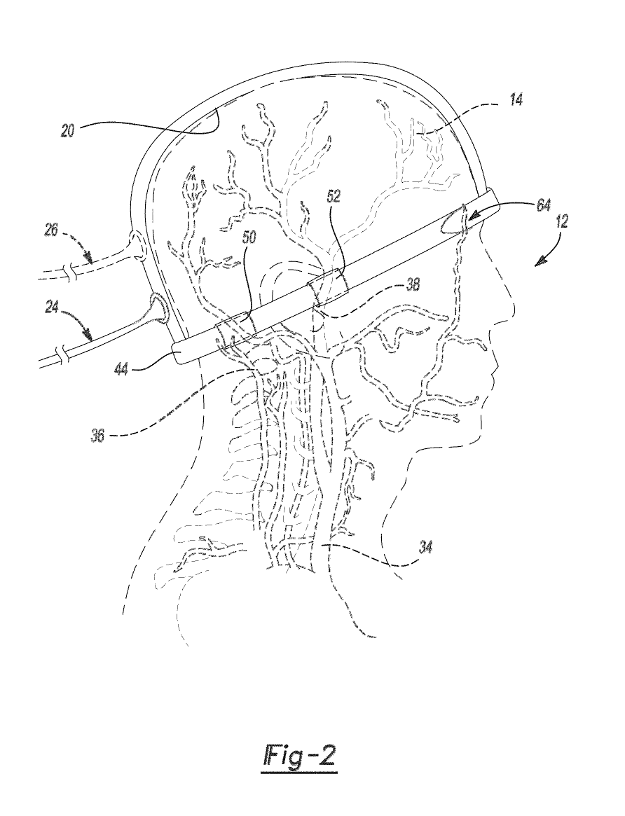

With reference to FIG. 1 and FIG. 2, a regulating device 10 is illustrated. The device 10 may include various portions, as discussed further herein, but is generally shaped and configured to be positioned on a head or scalp of a subject 12. The device 10 may be configured to appear as a cap for a human. For example, the device 10 may be positioned on a head or over a skull 14 of the patient 12. Generally the device 10 includes a reservoir system 16 formed between an exterior wall 18 and an interior wall 20. Generally the interior wall 20 may be formed to be positioned on the external skin or hair on the head of the patient 12.

The reservoir system 16 may be formed as a single volume container having no internal walls or features. It understood, however, that the reservoir system 16 may include internal baffles or connective members. The reservoir system 16 may be formed of a compliant material or flexible material such as selected polymers. Generally, the reservoir system 16 includes or forms a volume 22 between the two walls 18, 20. The volume 22 may be filled with a selected material that may act or operate as a heat transfer medium, such as a fluid. The heat transfer medium may include water, appropriate low freezing temperature solutions of water (e.g. a mixture of water and about 20% to about 70% glycerol), or other appropriate materials or other appropriate selected solutions. Whether filled or not a surface 23 of the wall 20 is generally formed to remain substantially in contact with the scalp 14 of the patient 12.

Selected ports may be made to the container 16, such as an inlet 24 and an outlet 26. The inlet 24 may include a connection or connector 28 and the outlet 26 may include a connection or connector 30. The connectors 28, 30 may connect to selected supply and return line, according to various embodiments, as discussed further herein. In various embodiments, only a single port, such as the port 24 may be provided. Thus, filling and emptying of the volume 22 may be performed through the single port.

For example, the single inlet line 24 may also be an outlet line where a user connects a supply to the connector 28 and fills the volume 22 with a selected material, such as the heat transfer medium discussed above. After a selected period of time, such as a selected set period of time or when the material in the volume 22 reaches a selected temperature, the line 24 may be used to empty the material from the volume 22 through the line 24. The user may then again connect a volume of material to the connector 28 and refill the volume 22 with a selected material. Accordingly, it is understood, that the container 16 may include only a single port as an inlet and outlet line that may be sequentially or serially used for either inlet or outlet depending upon a selected state of the material within the volume 22.

Schematically illustrated in FIG. 2 are arteries into the head 14 of the patient 12 that provide a blood supply to the head 14 of the patient 12. Generally, a blood supply is provided to at least a portion of the scalp or skull 14 through a carotid artery 34. The carotid artery 34 may branch into other arteries including an occipital artery 36 and a temporal artery 38. It is understood that other arteries may branch from the carotid artery 34, and they may also be engaged (e.g. compressed or restricted), as discussed herein, when selected.

The device 10 further includes a band or region 44. The reservoir system 16 may be fixed to and/or separate from the band, such as at a perimeter of the reservoir system 16. The band 44 may be formed of a selected material. The band may be formed of an elastic material and a selected size, thus will apply a selected and fixed amount of force to the head 14. The band 44 may also, and/or alternatively, be substantially or completely inelastic that is adjustable in length. The adjustable band 44 may provide for selecting a force applied to the head 14. It is understood, however, that the band 44 may be both elastic and adjustable. Regardless of the material the band 44 may be positioned and/or adjusted to apply a force, such as pressure, to the skin or dermis of the patient 12 relative to the skull 14.

The band 44 may include an elastic material and/or an adjustment member or assembly 64 (e.g. an adjustable buckle or strap) to adjust pressure or size of the band 44 relative to the patient 12. The adjustment of the band 44 allows the force applied, such as the pressure, of the band 44 and, therefore, the bumpers 50, 52 to be adjusted. In various embodiments, therefore, the pressure applied may be adjusted for patient comfort and/or selecting an amount of restriction of blood flow through the selected vessels. Generally the band 44, therefore, may apply pressure around the skull of the patient 12. In addition to the band 44 alone, one or more members, which may also be referred to as vascular pods, bumpers or pressure application members may be provided. The bumpers may include an occipital bumper 50 and a temporal bumper 52 that may be positioned relative to or on the band 44. The bumpers may be formed into or surround the band 44. Further, occipital bumpers 50 and temporal bumpers 52 may be provided on both a left and right side of the head 14. The bumpers 50, 52 may be an appropriate selected length, such as about 1 centimeters (cm) to about 8 cm, including about 4 cm to about 6 cm, and further about 5 cm (about 2 inches). The bumpers 50, 52 may be fixed or moveable relative to the band 44.

In various embodiments the bumpers 50, 52 may be formed as cylindrical members that are positioned on the band 44 and held in place relative to the skull 14. Further the bumpers 50, 52 may be substantially ridged members that are positioned between the band 44 and the skull of the patient 14 generally in the region of the selected arteries, such as the occipital artery 36 and the temporal artery 38. Nevertheless, by tightening or applying force with the band 44 to the patient 12, the regions where the bumpers 50, 52 are present will experience an increased pressure point.

Accordingly, an increased pressure or pressure points may be applied generally at or on top of the selected arteries, such as the occipital artery 36 and/or the temporal artery 38. In various embodiments, the bumpers 50, 52 may be sized, shaped, and positioned to apply pressure selectively to specific regions, such as vessels. Thus, the bumpers 50, 52 may be placed to apply pressure substantially only to the occipital artery 36 and/or the temporal artery 38. In this way, blood flow may be restricted through substantially only or entirely only the occipital artery 36 and/or the temporal artery 38.

By applying pressure to the arteries 36, 38, while not being bound by the theory, a reduction in blood flow through the arteries 36, 38 will occur due to mechanical pressure and restriction of size of the selected arteries 36, 38. By increasing the pressure and/or decreasing a circumference of the band 44, a force may be increased on the selected arteries 36, 38. In so doing blood flow may be limited to the skull 14 of the patient 12, at least for a selected period of time.

With continuing reference to FIGS. 1 and 2, the device 10 is configured to be positioned on the patient 12 and to allow for a material, such as the selected liquid, to be intermittently positioned or used to fill the volume 22 and removed from the volume 22. The introduction and removal of the material from the volume 22 may be based on a selected interval of time (e.g. 30 minutes), a selected measured temperature on the interior wall 20 of the cap 10 (e.g. about 2 degrees Celsius (C) to about 8 degrees C., including about 3 degrees C. (about 38 degrees Fahrenheit (F)) to about 7 degrees C. (about 45 degrees F.)), or other appropriate selected interval.

In various embodiments a thermal sensor 60 is positioned on the internal wall 20. The thermal sensor 60 may be any appropriate thermal sensor, such as a thermistor, or other selected thermal sensor. The thermal sensor 60 is configured to sense a temperature at the inner wall 20 of the device 10. The thermal sensor 60, therefore, may be used by a user to determine the temperature at the internal wall 20.

The thermal sensor 60 may send a signal to a control or display unit in a selected manner, such as wirelessly, wired, or other appropriate configuration. Wireless transmissions may include data transmission according to the Bluetooth® wireless data transmission protocols. The transmissions may be received by a receiver and used to display or evaluate the temperature at the inner wall 20. The user may then determine whether or not to refill the volume 22 based upon various protocols such as a selected range or threshold temperature of the inner wall 20, the remaining time of treatment, or other appropriate considerations.

The band 44, as discussed above, may include appropriate configurations to assist in providing a pressure or fit to the patient 12. In various embodiments a pull tab or other member may be used to cinch the band 44 onto the patient 12. In addition, other tightening or connection mechanisms 54 may be used to tighten the band 44 onto the patient 12. For example, the connection or tensioning mechanism 64 may include a ratcheting system that allows the band 44 to be ratcheted to a selected tightness or pressure on the patient 12 and/or released from the patient 12.

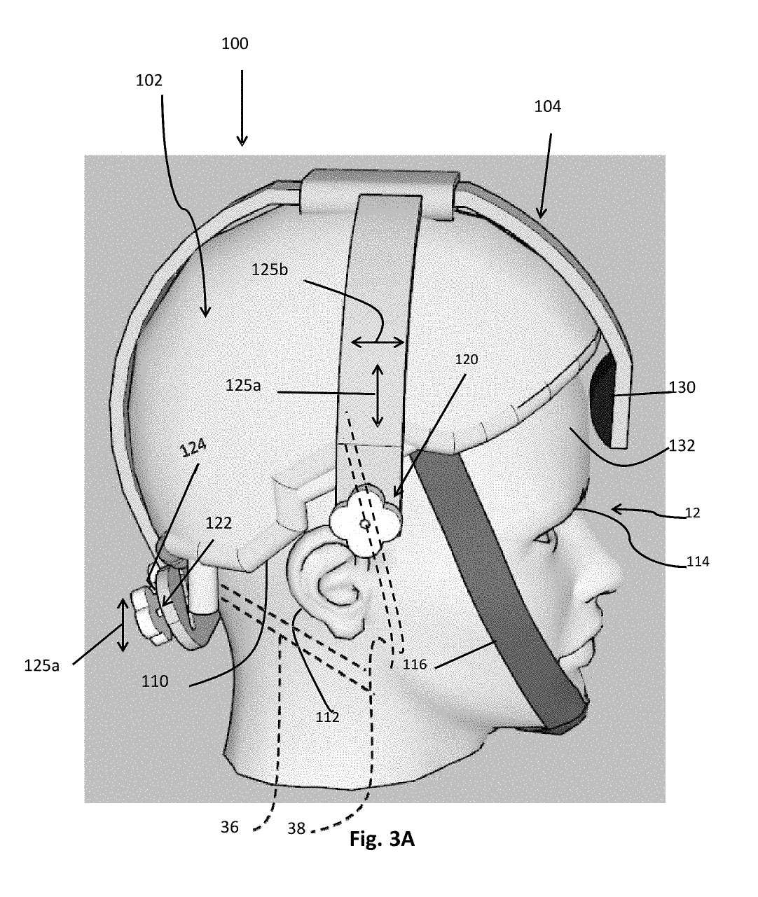

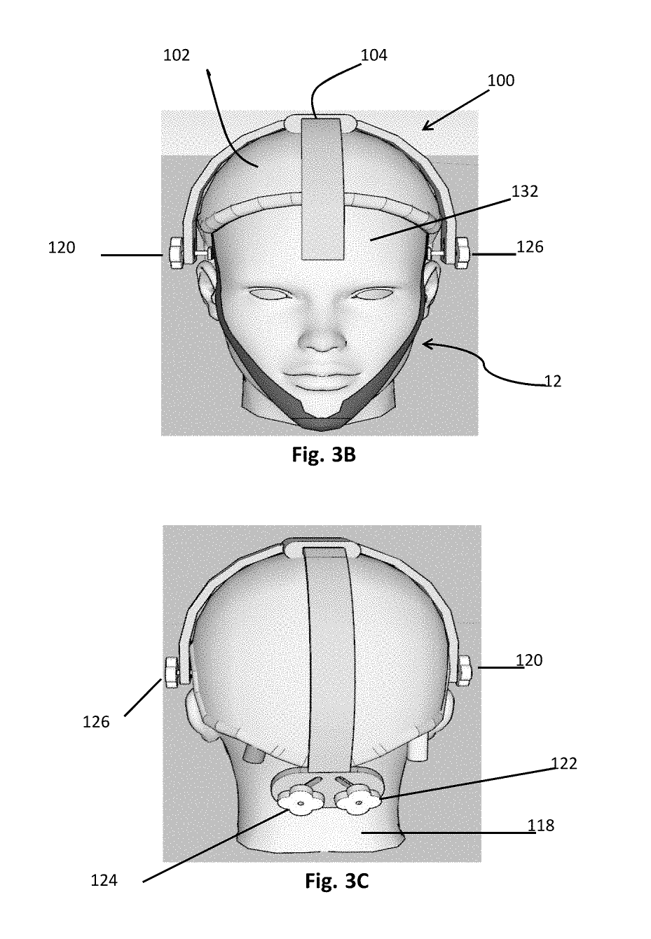

Turning reference to FIGS. 3A-9B a device 100 to regulate the head 14 of the patient 12 is illustrated. Certain features or portions of the device 100 are similar to those of the device 10. The device 100 includes various features and elements that are identical to or substantially similar to those of the device 10, and will not be repeated here. Various elements or features of the device 100, however, are in addition to or different than those illustrated and referenced with reference to the device 10. It is understood, however, that various features of the device 100 may be used in combination with the features and elements of the device 10, and vice versa.

The device 100 may include a thermal transfer system or assembly 102 and a pressure or bumper unit or assembly 104. The device 100, therefore, may include two separate portions, as discussed further herein, rather than having the thermal transfer portion 102 integrated as a single member with the pressure or bumper assembly 104. It is understood, therefore, similarly that the device cap 10 may also have the band 44 and the associated pressure application members separate from the cap 10 defining the volume 22. Nevertheless, they may also be integrated as a single unit. Accordingly the thermal transfer assembly 102 separate from the pressure application assembly 104 in the cap 100, is merely exemplary.

The thermal transfer unit 102 is positioned on the patient 12, as exemplarily illustrated in FIGS. 3A-3C, so as to cover at least a portion of the scalp or head 14, similar to the device 10. The thermal transfer unit 102 may include a proximal edge 110 that has a selected shape or contour to fit over various portions of the anatomy of the subject 12, such as an ear 112, eyebrows 114, or other anatomical features of the patient 12. Further, a retention strap or member 116 may be attached to a portion of the thermal transfer unit 102 to hold the thermal transfer unit 102 onto the patient 12 for a selected period of time. The retention strap 116 may be a soft elastic material to provide a gentle tension or snug fit of the thermal transfer unit 102 onto the patient 12.

The thermal transfer unit 102 may be shaped and configured to extend over a selected portion of the patient 12, such as a distance over the forehead or anterior portion of the head 14 and extending generally to near a base of the skull near or at the occipital bone and/or near the neck 118.

Generally the thermal transfer unit 102 may include various thermal transfer elements, as discussed further herein, to assist or provide a means of reducing or transferring heat or thermal energy from a surface or portion of the patient 12 away therefrom, such as with a flow of a heat transfer medium (e.g. heat transfer fluid), as discussed further herein.

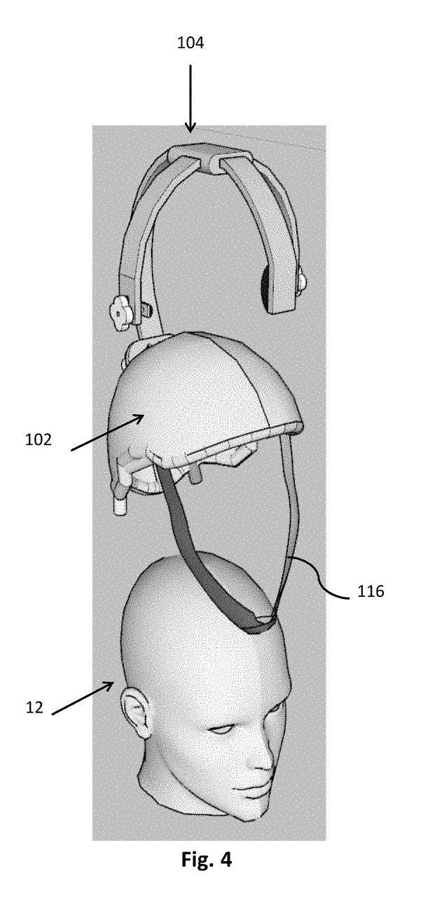

As illustrated in FIG. 4, the thermal transfer unit 102 is configured to be positioned on the patient 12 and may be positioned on the patient 12 separate from the pressure application unit 104. Generally, the thermal transfer unit 102 may be positioned on the patient 12 and at least temporarily secured in place with the tension strap 116. The bumper unit 104 may then be positioned onto the patient 12, such as over the thermal transfer unit 102, as illustrated in FIGS. 3A-3C. The device 100, therefore, may be positioned on the patient 12 in a stepwise manner to assist in ensuring a selected position of the thermal transfer unit 102 on the patient 12 and of the bumper unit 104 separate therefrom. As discussed herein, positioning of the bumper unit 104 separate from the thermal transfer unit 102 may allow for a difference between selected patients and an optimization of positioning the bumper unit relative to the patient 12, such as relative to the selected arteries as discussed above and herein.

Returning reference to FIGS. 3A-3C, the bumper unit 104 may include various individual units or pods, which may also be referred to as vascular or arterial pods or bumpers, such as a first temporal artery bumper unit 120, a first occipital artery bumper unit 122, a second occipital artery bumper unit 124, and a second temporal artery bumper unit 126. Each of the artery bumper units 120-126 may be selectively moved to apply pressure to the selected temporal and occipital arteries 38, 36 of the patient 12. An increased pressure or pressure points may be applied generally at or on top of the selected vessels, such as the occipital artery 36 and/or the temporal artery 38 with the bumper unit 104 including the bumper units 120-124. Further, the bumpers may be adjusted in the directions of arrows 125a, 125b (as illustrated in FIG. 3A) to align the respective bumpers over the appropriate occipital artery 36 and/or the temporal artery 38. In various embodiments, the bumper units 120-124 may be sized, shaped, and positioned to apply pressure selectively to specific regions, such as vessels. Thus, the bumper units 120-124 may be placed to apply pressure substantially only to the occipital artery 36 and/or the temporal artery 38. In this way, blood flow may be restricted through substantially only or entirely only the occipital artery 36 and/or the temporal artery 38.

A positioning or counterbalance bumper or member 130 may also be formed with the pressure unit 104 to contact a portion of the patient 12, such as a forehead 132 of the patient 12. The counterbalance unit 130 may assist in holding the bumper unit 104 relative to the patient 12 during the application of the pressure to the selected arteries, such as the temporal artery 38 and the occipital arteries 36. Generally, the bumper unit 104, therefore, may apply pressure to the selected arteries to assist in constricting or applying a mechanical pressure to the arteries similar to the band 44 and respective bumpers 50, 52 as discussed in relation to the cap 10.

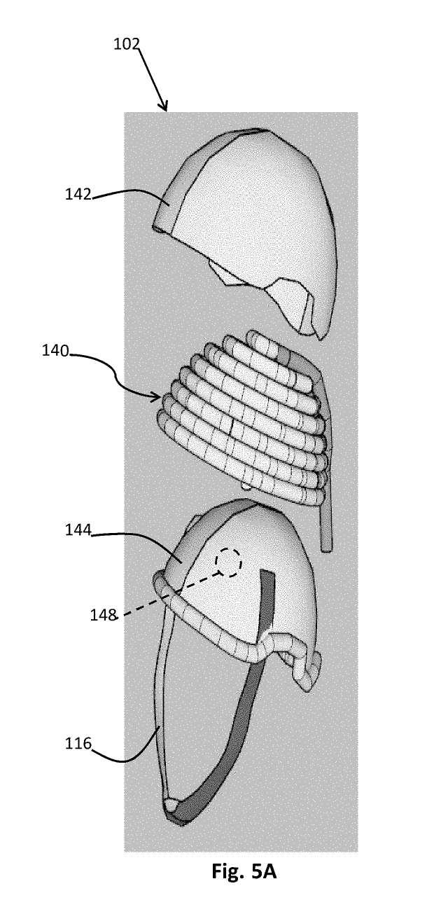

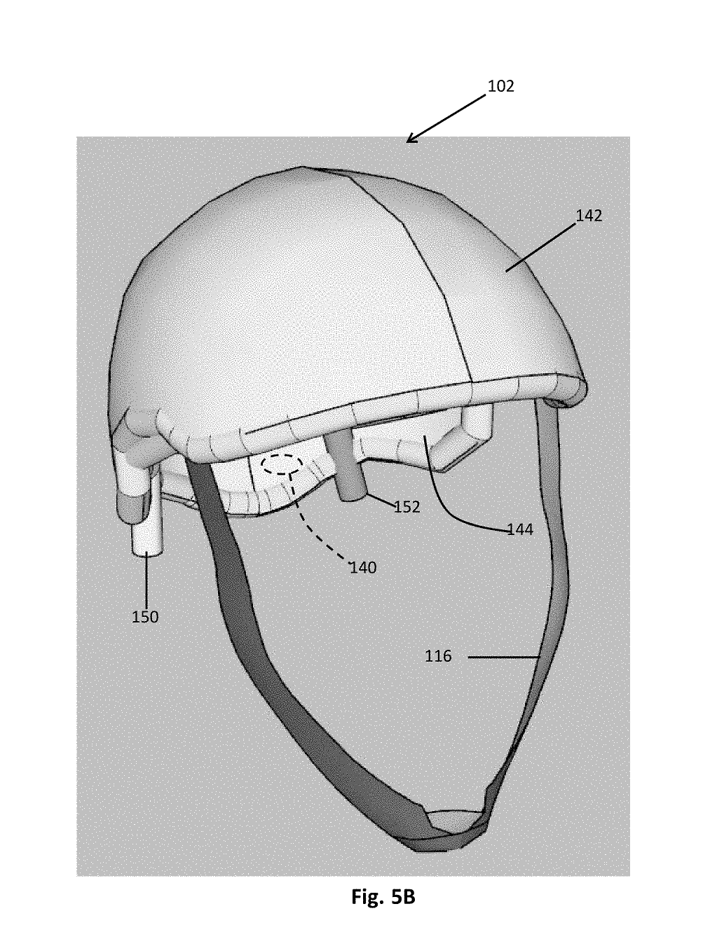

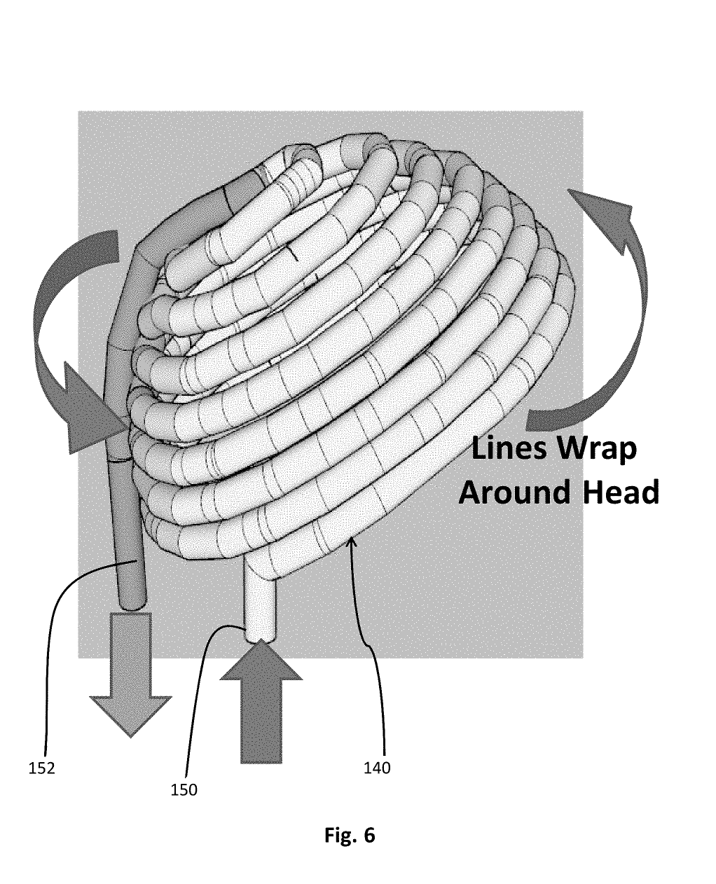

With continuing reference to FIGS. 3A-4 and additional reference to FIGS. 5 and 6, the cooling or thermal transfer unit 102 is discussed in greater detail. The thermal transfer unit 102 may include a fluid transfer line or circulation line 140. The line 140 may include a tubing of selected materials and wall thickness. In various embodiments, it is understood that the fluid transfer line 140 may be substantially the only element of the thermal transfer unit 102. For example, the thermal transfer line 140 may be placed directly on the patient 12 and the pressure or bumper unit 104 may be positioned over the fluid transfer lines 140. For various reasons, such as ease of use and/or cleaning, the thermal transfer unit 102 may include a cover 142 and a head or contact layer or wall 144. The tension strap 116 may be connected to the inner or second wall 144.

Further, as discussed above, a thermal sensor 148 may be positioned on the inner or second wall 144. Again the thermal sensor 148 being near the patient 12 may assist in determining a temperature at a surface of the patient's skin or other appropriate region. It is understood, however, that the thermal sensor 148 may also be positioned on the fluid transfer lines 140. In various embodiments the thermal transfer lines 140 may be positioned between the first all 142 and the second wall 144, such as in a manufacturing phase, and the two walls 142, 144 may be connected together. For example, the two walls 142, 144 may be formed with a selected flexible material or textile and may be at least partially stitched together to hold the two walls 142, 144 together.

The thermal transfer line 140 may include a length of tubing that is formed as a single piece and/or connected pieces. Regardless the thermal transfer tubing 140 may include a single inlet 150 and a single outlet 152 that are fluidly connected by the line 140. The heat transfer medium, such as a heat transfer fluid, may then be delivered through the thermal transfer tubing 140 by delivering it and passing it through the inlet 150 and allowing it to exit through the outlet 152. As the fluid circulates through the tubing 140 heat may be transferred from the patient 12 to the fluid and then the heated fluid may be passed out of the tubing 140 through the outlet 152. It is understood that the fluid may be any appropriate fluid such as those discussed above. The heat transfer fluid may include liquid fluids, gaseous fluids, or materials that change states within the tubing to assist in efficient or rapid cooling, such as a liquid that turns to a gas within the tubing 140. Regardless the fluid may transfer through the tubing 140 from the outlet 152.

Turning reference to FIGS. 7, 8, and 9A-9B, and with continuing reference to FIGS. 3A-3C, the bumper or pressure unit 104 will be described in greater detail. The bumper unit 104, as illustrated in FIG. 7, may include various arms or limbs 160, 162, 164, and 166 that extend from a central hub or connection region 170. It is understood that the limbs 160-166 and the connection area 170 may be formed as a single member, such as by injection molding or other molding process to form a biasing or holding structure 172 substantially defined by the four limbs 160-166 and the central hub or connection 170. It is further understood that having individual limbs is not a requirement, and rather that the holding or biasing unit 172 may be formed as a dome, such as semispherical or hemispherical to fit the head 14 of the patient 12. In various embodiments, however, the individual limbs 160-166 may allow for greater adjustability of the biasing member 172.

The arms 160-166 may extend from the central hub or connection 170 in generally an arced or curved manner or shape to respective terminal ends 160a-166a. The arms 160-166 may be adjustable in length relative to the central hub 170, such as by sliding relative to the hub 170. Alternatively, or in addition thereto, the unit 172 may be formed in different sizes such as the length to the arms 160-166 from the hub 170 may be selected in different sizes.

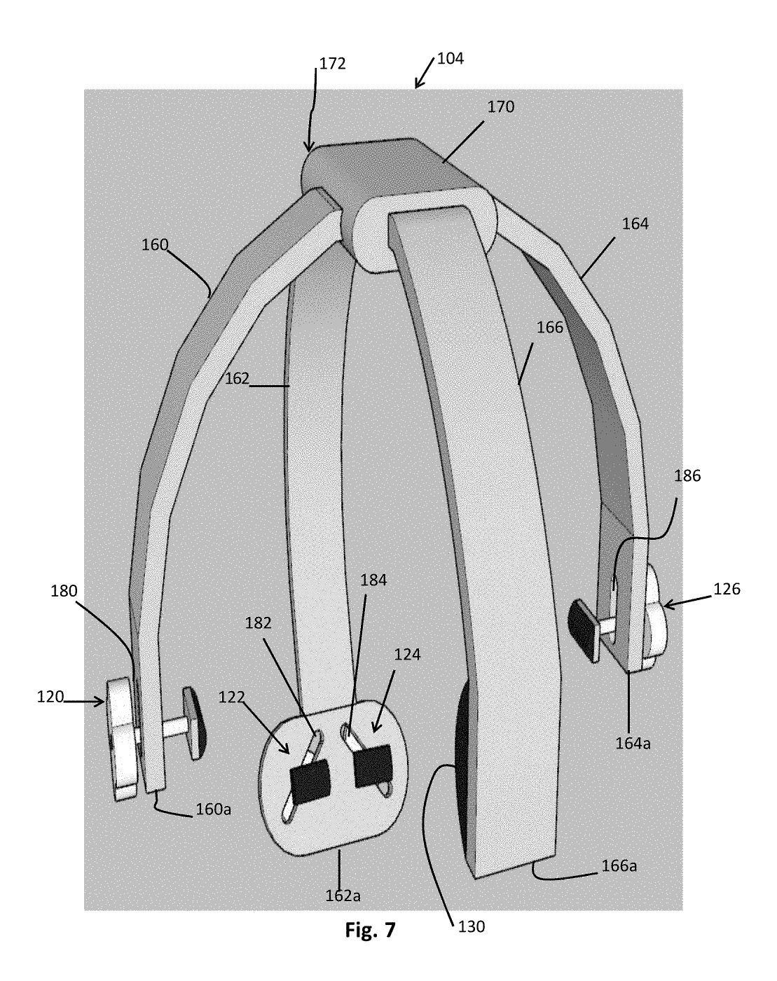

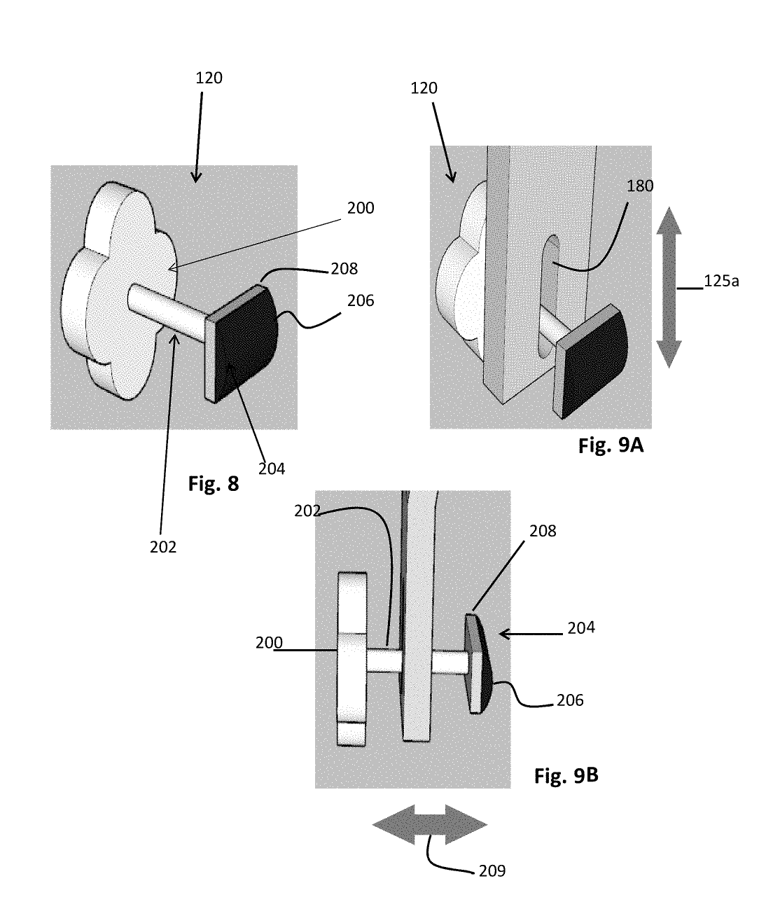

Nevertheless, near each of the ends 160a, 162a, and 164a are one or more of the adjustable bumpers or artery bumper units 120-126, as discussed above. Each of the artery bumper units 120-126 are positioned relative to a through-bore such as a first through-bore 180, a second through-bore 182, a third through-bore 184, and a fourth through-bore 186. The through-bores 180-186 may be formed in any appropriate configuration, such as substantially elongated slots. For example, slots may be elongated generally along a longitudinal axis of the patient 12. Therefore the artery bumper units 120-126 may move along a long axis of the through-bore, generally along the double headed arrow 125a as illustrated in FIG. 9A. As an example only, the compression unit 120 is illustrated in FIGS. 8-9B and passes through the through-bore 180. It is understood that the through-bores may be positioned at an angle relative to the longitudinal axis of the patient 12, such as an angel thereto for example for the through-bores 182 and 184.

Each of the artery bumper units 120-126 may include substantially identical features, including those illustrated in FIGS. 8-9B. Accordingly, the discussion of the first bumper unit 120 and the features thereof may be included in the other bumpers 122-126 but will not be repeated.

According to various embodiments, the artery bumper unit 120 may include a handle or manipulation member 200. A shaft 202 may extend from the handle 200 and engage a bumper assembly 204. The bumper assembly 204 may include a bumper member 206 that is connected to a holding or fixed portion 208. It is understood that the bumper portion 204 may be formed as a single member and having two members is merely exemplary. Nevertheless, the handle 200 may be threaded, such as having an internal thread to engage an external thread of the shaft 202. By turning the handle 200 relative to the shaft 202 the handle 200 may move axially along the shaft generally in the direction of arrow 209. By moving the handle 200 along the shaft 202, the bumper assembly 204 may also move generally in the direction of arrow 209 to allow adjustment and movement of the bumper assembly 204 for selected purposes, as discussed herein. Therefore, the bumper unit 204 may move closer to the patient to increase a force (e.g. pressure) on the patient 12, such as on the temple artery 38. Twisting the handle 200 in an opposite direction may move the shaft or handle in an opposite direction of the arrow 209 and decrease the pressure on the artery, such as the temple artery 38. The shaft 202 may be fixed to the bumper assembly 204, such as the second member 208. The adjustment of the bumper units, such as unit 120, in the direction of arrow 209 may, therefore, be used to apply and/or adjust the application of force applied to the patient 12, such as the vessels. In various embodiments, therefore, the pressure applied may be adjusted for various purposes, such as patient comfort and/or selecting an amount of restriction of blood flow through the selected vessels, including the occipital and/or temporal arteries 36, 38. It is further understood, the patient 12 and/or a user may adjust the bumper units. Further, each of the bumper units may be adjusted separately to allow for a selected force applied by each bumper. Although, it is understood, that all of the bumpers may be adjust in a single manner.

In various embodiments the handle 200, shaft 202, and holding portion 208 of the bumper assembly may be formed of a substantially ridged material, such as a selected metal or rigid polymer. This may provide rigidity and stiffness to allow for pressure to be applied to the subject 12. The bumper portion 206 may be formed of a selected material that may be softer than the shaft 202 and/or the handle 200 to apply a gentle and selected pressure to the patient 12. For example, the bumper member 206 may be formed of rubber, gel, sponge, or an encapsulated air volume or air chamber. For example, a soft rubber or gel may be provided to form the bumper member 206. Alternatively, or in addition thereto, an air-chamber may be provided as the bumper member 206. The bumper member 206 may have a selected plasticity or bending modulus that provides force to the selected region, such as the vessel, to reduce a blood flow there through (such as about reduction of about 10% to about 80%, including about 40% to about 60%) without causing trauma to the surrounding soft tissue or death or damage to the vessel.

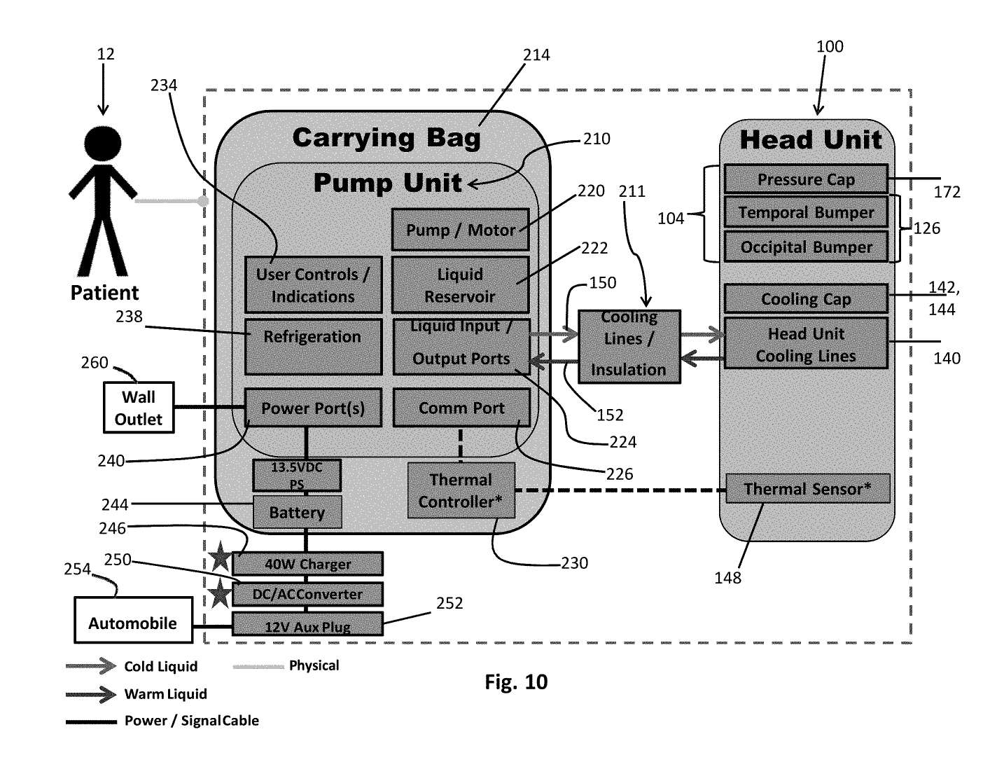

With reference to FIG. 10, the device 100 may be connected to a powered delivery system or assembly usable by the patient 12. As schematically illustrated in FIG. 10, the device 100 may include the pressure or bumper assembly 104 including the arm and hub unit 172 and the respected bumpers 120-126. The device 100 may further include the shell portions 142, 144 and the flow or transfer lines 140. The inlet 150 and the outlet 152 may extend from the device 100 along supply lines or tubing 2111. The supply lines 211 may be insulated with selected insulation. The lines 2111 may connect to a pump assembly or unit 210 that may be in a selected location or may be substantially portable in a carry or portable bag 214.

The pump unit 210 may include various portions such as a pump motor 220 that can pump the heat transfer medium, such as a fluid from a liquid or fluid reservoir 222. Inlets and outlets may be associated with the reservoir 222 such as inlets and outlets 224. The inlets and outlets 224, through the lines 211, may be connected to respective inlet 150 and outlet 152 of the device 100, or other appropriate connections of the device 10.

The pump unit 210 may have various communication ports, such as a communication port 226 to communicate with a control or a sensor, such as a thermal controller 230 that may be connected with the selected or optional thermal sensor 148. The pump unit 210 may be operated based upon instructions regarding a temperature threshold which may be measured by the thermal sensor 148 and communicated or monitored by the thermal controller 230 through the communication (comm) unit 226 to a control unit 234. The control unit 234 may be any appropriate selected controller such as an application specific integrated circuit, a general purpose programmable processor, or other appropriate controller. The control unit 234 may also have an external control or user information system. For example, a separate control or information display may receive a signal from the control unit 234 regarding operation and/or transmit a signal to the control unit 234 regarding operational parameters (e.g. temperature range, flow rate, etc.). The communication with the control unit 234 may be from a selected system (e.g. hand held device) and using appropriate and selected communication protocols (e.g. Bluetooth® wireless communication protocols).

The pump unit 210 may include an optional refrigeration assembly 238 to allow regenerative cooling of the fluid in the reservoir when transmitted to the pump unit 210 from the device 100. Refrigeration or regeneration of the cooling or thermal transfer medium, however, may be done outside of the pump unit 210. Further, a constant supply of medium at a selected temperature may be provided from a source separate from the pump unit 210.

Power may be provided to the pump unit 200 through various power inlets 240 such as through a battery 244 that may be charged with selected chargers, including a 40 watt charger 246 connected to various converters such as a DC-AC convertor 250 that may convert direct current power through a selected plug, such as a 12 volt auxiliary power supply 252 in an automobile 254. Selected other power may also be direct from a selected AC converter, such as an all outlet/converter 260. Regardless the pump unit 210 may be powered at a selected rate to power the pump to pump the fluid through the lines to the inlet 150 to the device 100 and from the device 100 through the outlet line 152 to allow regenerative cooling of the fluid within or for transfer to the device 100.

The pump unit 210 may be moved by the patient 12 and/or other user. In various embodiments, the pump unit 210 may be provided with the carry bag 214 to ease and efficient transport. It is understood, however, that the pump unit may be provided with any appropriate transfer system.

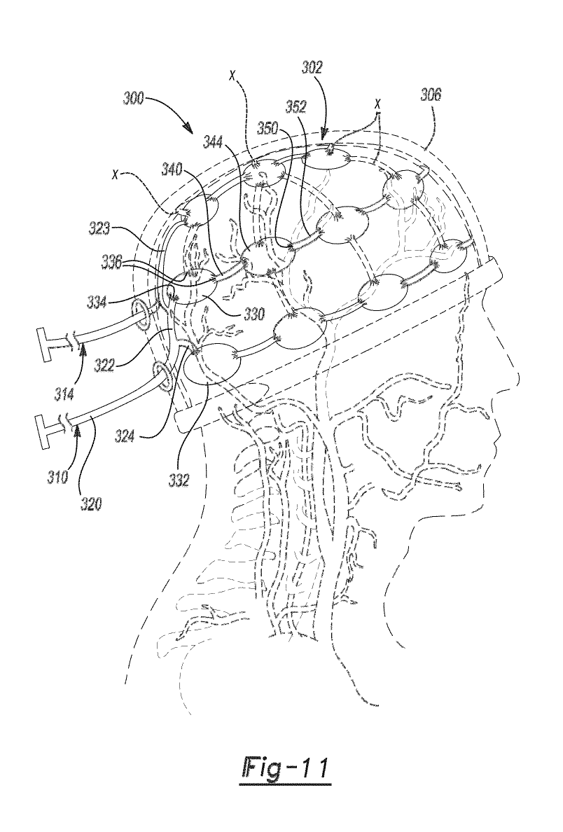

With reference to FIG. 11, a device assembly 300 is illustrated. The device assembly 300 includes a transfer or circulation assembly 302. The transfer assembly 302 may be used to circulate or transfer the heat transfer medium, as discussed above.

The transfer assembly 302 may be placed directly on the head 14 or on a first layer, similar to the layer 144 discussed above. Similarly a cover or optional over layer 306 may be positioned over the fluid circulation assembly 302. The cover 306 may include an elastic or lightly compressive construction to assist in holding the fluid circulation assembly 302 in place on the head 14. Although not illustrated in FIG. 11, the compression or pressure assembly 104 may also be positioned over the fluid transfer circulation assembly 302 to assist in mechanically compressing or constricting arteries in the patient 12, similar to as illustrated in FIGS. 3A-3C.

The fluid flow assembly 302 may include an inlet 310 and an outlet 314. The inlet and outlet may be a single port, such as only the inlet 310 or outlet 314 that are connected in a continuous web or connection of lakes or bags, which may also be referred to as anastomosis. Generally, the fluid flow assembly may include a first or main line 320 extending from the inlet 310. The main or first line 320 may branch into at least two or more lines (e.g. secondary lines) 322 and 324. Each of the branch lines 322, 324 may enter an anastomosis bag or region, respectively, 330 and 332.

The branch lines further branch into smaller branch lines or constructions (e.g. tertiary or capillary lines) 334 as it enters or within the bag 330. The tertiary branches 334 may be formed to have a selected small internal diameter, as discussed herein. At a second end or side of the bag 330 may be a second grouping of capillaries lines 336. These may then combine into a secondary branch or connection 340. Again the secondary branch 340 may connect or extend to an additional bag 344. At a second side of the bag 344 may be a further assembly or construction of capillary lines 350 that are fluidly connected to a subsequent secondary line 352.

As illustrated in FIG. 11, it is understood that a plurality of the anastomosis bags may be positioned in a fluidly connected network or spaced apart configuration. Each of the secondary lines may lead to one of the bags 330 and in the bag formed as a plurality of small tubes or capillaries having a selected diameter. Generally the diameter of the small tubes, such as the capillaries 334 may be substantially less, such as about 10% to about 0.01% a diameter of the secondary or tertiary lines, such as the secondary line 322. The network that is fluidly connected may be seized to cover a selected portion of the head 14, as exemplary illustrated in FIG. 11.

The pressure of a fluid entering any of the bags, such as the bag 330, on one side may or will cause fluid to flow through the bag 330 to the capillaries 336 on the second side and throughout the network to the outlet 314. The transfer assembly 302, therefore, will allow a transfer of a fluid throughout the assembly 302. In various embodiments, the device 300 may be provided where each of the bags 330 have only two connections or ports (such as an inlet and an outlet) and only one connection to any one other of the bags 330. In this configuration, referred as “in series”, the bags 330 are more able to have a consistent and equal or even flow regardless of the position on the patient 12. It is understood, however, that more than two connections from each of the bags 330 may be provided (illustrated in phantom lines (exemplarily “x”) in FIG. 11). With multiple connections a lattice or parallel configuration is achieved. Regardless, it may be selected to have the single include 310 and single outlet 314.

The bags, such as the bag 330, may be positioned at selected locations or positions on the patient 12 to assist in having a high flow rate and/or high volume at a specific and selected locations. The high flow rate may increase a cooling effect or thermal conduction in the specific selected location. In this way the device 300 could be provided to provide a selected, such as high, cooling effect at specific locations on the head of the patient 12. In this way the user could selectively position areas that would be cooled greater than other areas on the patient 12.

It is further understood that the various transfer lines may be molded as shapes or as passages and bags (e.g. volumes) in a solid structure. For example, rather than the cover 306 being separate from the cooling or fluid flow assembly 302, the lines (e.g. the secondary lines 322 the capillaries 334) and the bag 330 could be molded into a solid or unitary member with the inlet 310 and the outlet 314 extending therefrom. Therefore although the fluid flow assembly 302 is illustrated as a connection of the bags 330 and selected tube sizes in diameters, it is understood that they may be formed as a channels and voids in a single member.

Further the fluid flow assembly 302 may be connected to the pump unit 210, as discussed above in FIG. 10. The pump unit 210 including the inlet and outlet connections 224 may be connected to the inlet and outlet 310, 314, respectively, of the fluid transfer unit 302. Therefore the fluid transfer unit 302 may also be connected to the pump unit 210 and operated as discussed above.

According to various embodiments, therefore, as discussed above, a device may be provided to regulate a portion of a subject. The regulation may include cooling and/or limiting blood flow to a portion of the subject. The regulation may allow for selected results or reduction in side effects, such as reduction in hair loss or elimination of hair loss, of the subject. Moreover, as discussed above, the bumpers, according to various embodiments, may be provided to apply a force (e.g. pressure) to only or substantially only selected arteries, occipital and/or temporal arteries. In various embodiments, therefore, the pressure applied may be adjusted for various purposes, such as patient comfort and/or selecting an amount of restriction of blood flow through the selected vessels, including the occipital and/or temporal arteries 36, 38. It is further understood, the patient 12 may adjust the bumper units and/or the user. Further, each of the bumper units may be adjusted separately to allow for a selected force applied by each bumper. Although, it is understood, that all of the bumpers may be adjust in a single manner.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for regulating a portion of a subject, the method comprising:
an elongated member configured to extend relative to a head of a subject;
a bumper member held with the elongated member relative to the head of the subject; and
a reservoir configured to hold a volume of a fluid relative to the head of the subject;
wherein the bumper member is selectively pressed against the head of the subject.

2. The system of claim 1, wherein the elongated member is adjustable in length to increase or decrease a pressure applied to the head of the subject.

3. The system of claim 2, wherein the bumper member is sized and shaped to press on an artery within the head of the subject.

4. The system of claim 3, wherein the bumper member includes a plurality of bumper members;
wherein each bumper member of the plurality of bumper members is positioned on the head of the subject to apply pressure to the temporal artery or the occipital artery.

5. The system of claim 1, wherein the reservoir includes an access port;
wherein the access port is configured to allow removal and replacement of the fluid within the reservoir.

6. The system of claim 5, further comprising:
a pump to automatically and/or continuously exchange the fluid in the reservoir.

7. The system of claim 6, wherein the access port includes an inlet access port and an outlet access port;
wherein the reservoir is configured to be emptied through the outlet access port and filled through the inlet access port.

8. The system of claim 1, wherein the elongated member includes:
a first arm; and
a second arm;
wherein the bumper member includes a plurality of bumper members;
wherein the first arm includes a first bumper member moveably connected thereto and the second arm includes a second bumper member moveably connected thereto.

9. The system of claim 8, wherein the first arm is configured to position the first bumper member relative to a temporal artery and the second arm is configured to position the second bumper member relative to an occipital artery.

10. The system of claim 8, further comprising:
a third arm; and
a fourth arm;
wherein the third arm includes a third bumper member and a fourth bumper member both moveably connected thereto.

11. The system of claim 10, wherein the first arm is configured to position the first bumper member relative to a left temporal artery, the second arm is configured to position the second bumper member relative to a right temporal artery, and the third arm is configured to position the third bumper member relative to a right occipital artery and the fourth bumper member relative to a left occipital artery.

12. The system of claim 1, wherein the reservoir includes:
a main line;
a secondary inlet line branching from and in fluid connection with the main line;
a plurality of capillary inlet lines extending from and in fluid connection with the secondary line;
a bag surrounding the plurality of capillary inlet lines a plurality of capillary outlet lines in fluid connection with the bag; and
a secondary outlet line in fluid connection with the plurality of capillary outlet lines;
wherein the fluid is able to flow into the main line, secondary inlet line, plurality of capillary inlet lines, the bag, the plurality of capillary outlet lines, and the secondary outlet line.

13. The system of claim 1, further comprising:
a pump to automatically and/or continuously exchange the fluid in the reservoir.

14. The system of claim 13, further comprising:
a thermal sensor to sense a temperature at least at a surface of the head of the subject.

15. The system of claim 14, further comprising:
a controller configured to control the pump to pump fluid through the reservoir based on instructions regarding a temperature sensed with the thermal sensor.

16. A system for regulating a portion of a subject, the method comprising:
an elongated member configured to extend relative to a head of a subject;
a bumper member held with the elongated member relative to the head of the subject, wherein the bumper member is selectively pressed against the head of the subject; and
a reservoir configured to hold a volume of a fluid relative to the head of the subject;
wherein the reservoir includes,
a main line;
a secondary inlet line branching from and in fluid connection with the main line;
a plurality of capillary inlet lines extending from and in fluid connection with the secondary line;
a bag surrounding the plurality of capillary inlet lines a plurality of capillary outlet lines in fluid connection with the bag; and
a secondary outlet line in fluid connection with the plurality of capillary outlet lines;
wherein the fluid is able to flow into the main line, secondary inlet line, plurality of capillary inlet lines, the bag, the plurality of capillary outlet lines, and the secondary outlet line.

17. A system for regulating a portion of a subject, the method comprising:
a reservoir configured to hold a volume of a fluid relative to the head of the subject, wherein the reservoir includes a single tube having an inlet port and an outlet port and configured to be positioned on a head of a subject;
a bumper assembly having,
a first arm having a first bumper member moveably connected thereto,
a second arm having a second bumper member moveably connected thereto,
a third arm having a third bumper member and a fourth bumper member both moveably connected thereto, and
a fourth arm,
wherein the first arm is configured to position the first bumper member relative to a left temporal artery, the second arm is configured to position the second bumper member relative to a right temporal artery, and the third arm is configured to position the third bumper member relative to a right occipital artery and the fourth bumper member relative to a left occipital artery.

18. A method of limiting hair-loss in a subject, comprising:
compressing at least one artery in a head of the subject during a treatment period; and
regulating a temperature of the head of the subject with a heat transfer medium.

19. The method of claim 18, wherein compressing at least one artery in the head of the subject during the treatment period includes:
placing a bumper member over an artery in the head of the subject; and
applying a force to the bumper member operable to restrict a flow of blood through the artery.

20. The method of claim 18, wherein compressing at least one artery in the head of the subject during the treatment period includes:
placing a first bumper member over an occipital artery in the head of the subject;
applying a first force to the first bumper member operable to restrict a flow of blood through the occipital artery;
placing a second bumper member over a temporal artery in the head of the subject;
applying a second force to the second bumper member operable to restrict a flow of blood through the temporal artery.

21. The method of claim 18, wherein regulating the temperature of the head of the subject with the heat transfer medium includes exchanging a first volume of the heat transfer medium with a second volume of the heat transfer medium when the first volume reaches a selected temperature.

22. The method of claim 21, wherein exchanging the first volume of the heat transfer medium with the second volume of the heat transfer medium when the first volume reaches the selected temperature includes pumping the heat transfer medium through a reservoir.

23. The method of claim 22, further comprising:
placing the reservoir on the head of the subject.
